(19) United States
(12) Patent Application Publication
(10) Pub. No.: US 2024/0181387 A1
(43) Pub. Date: Jun. 6, 2024

(54) OXYGEN CONCENTRATION MODULE

(71) Applicant: Skyre, Inc., East Hartford, CT (US)

(72) Inventors: Trent M. Molter, Suffield, CT (US); Lawrence Moulthrop, Windsor, CT (US); Thomas Maloney, Hebron, CT (US); Karen Murdoch, Somers, CT (US); Gail Brackett, Los Angeles, CA (US)

(21) Appl. No.: 18/570,882

(22) PCT Filed: Jun. 16, 2022
(86) PCT No.: PCT/US2022/033799

§ 371 Date: Dec. 15, 2023

Related U.S. Application Data
(60) Provisional application No. 63/211,248, filed on Jun. 16, 2021.

(51) Int. Cl. B01D 53/32; A61M 16/10; B01D 53/26

(52) U.S. Cl. CPC B01D 53/326; A61M 16/101; B01D 53/26; B01D 2256/12; B01D 2257/80

(57) ABSTRACT

In an aspect, the oxygen concentrator module can comprise an electrochemical cell (40) comprising a cathode (34), an anode (54), a proton exchange membrane (42) located in between the cathode (34) and the anode (54), a cathode side chamber (32) located on a side of the cathode (34) opposite the proton exchange membrane (42), and an anode side chamber (52) located on a side of the anode (54) opposite the proton exchange membrane (42); a gas feed stream (10) in fluid communication with the cathode side chamber (32); a concentrated oxygen stream (56) in fluid communication with the anode side chamber (52) to remove the concentrated oxygen stream (56) from the anode side chamber (52); a separated water stream (96) in fluid communication with the cathode side chamber (32) to remove the separated water stream (96) from the cathode side chamber (32); and an enthalpy exchanger (20) in fluid communication with the cathode side chamber (32) via an exchanged stream (22), wherein the gas feed stream (10) is in fluid communication with the enthalpy exchanger (20) upstream of the electrochemical cell (40) to form the exchanged stream.

### CROSS-REFERENCE TO RELATED APPLICATION

This application claims the benefit of U.S. Provisional Patent Application Ser. No. 63/211,248 filed Jun. 16, 2022. The related application is incorporated herein in its entirety by reference.

### BACKGROUND

In such instances, the easy production of highly concentrated oxygen is important. For example, for patients that are struggling to breathe, it can be very difficult to provide them with sufficient amounts of oxygen. Hospitals and care facilities often keep pressurized oxygen tanks on hand to provide oxygen to patients as needed, but these tanks can be cumbersome to move and many safety precautions must be taken during storage and transport.

Improved systems for concentrating oxygen on demand are therefore needed.

### BRIEF SUMMARY

Disclosed herein is in an oxygen concentrator module.

In an aspect, the oxygen concentrator module can comprise an electrochemical cell 40 comprising a cathode 34, an anode 54, a proton exchange membrane 42 located in between the cathode 34 and the anode 54, a cathode side chamber 32 located on a side of the cathode 34 opposite the proton exchange membrane 42, and an anode side chamber 52 located on a side of the anode 54 opposite the proton exchange membrane 42; a gas feed stream 10 in fluid communication with the cathode side chamber 32; a concentrated oxygen stream 56 in fluid communication with the anode side chamber 52 to remove the concentrated oxygen stream 56 from the anode side chamber 52; a separated water stream 96 in fluid communication with the cathode side chamber 32 to remove the separated water stream 96 from the cathode side chamber 32; and an enthalpy exchanger 20 in fluid communication with the cathode side chamber 32 via an exchanged stream 22, wherein the gas feed stream 10 is in fluid communication with the enthalpy exchanger 20 upstream of the electrochemical cell 40 to form the exchanged stream.

In another aspect, a method of concentrating oxygen comprises introducing a gas feed stream 10 to an cathode side chamber 32 of an electrochemical cell 40 comprising a cathode 34, an anode 54, a proton exchange membrane 42 located in between the cathode 34 and the anode 54, the cathode side chamber 32 located on a side of the cathode 34 opposite the proton exchange membrane 42, and an anode side chamber 52 located on a side of the anode 54 opposite the proton exchange membrane 42; removing a concentrated oxygen stream 56 from the anode side chamber 52; removing a separated water stream 96 from the cathode side chamber 32; and directing the gas feed stream 10 to an enthalpy exchanger 20 upstream of the electrochemical cell 40 and hydrating the gas feed stream 10 in the enthalpy exchanger.

The above described and other features are exemplified by the following figures, detailed description, and claims.

### BRIEF DESCRIPTION OF THE DRAWINGS

The following Figures are exemplary embodiments, which are provided to illustrate the present disclosure. The figures are illustrative of the examples, which are not intended to limit devices made in accordance with the disclosure to the materials, conditions, or process parameters set forth herein.

FIG. 1 is an illustration of an aspect of an oxygen concentrator module;

FIG. 2A is an illustration of an aspect of an oxygen concentrator module having an anode side water feed;

FIG. 2B is an illustration of an aspect of a gas feed introduction unit; and

FIG. 3 is an illustration of an aspect of an oxygen concentrator module with a cathode side water feed.

### DETAILED DESCRIPTION

An Oxygen Concentrator Module (OCM) has been developed that can use an electrochemical process to produce, store, and administer concentrated oxygen to a patient without requiring an external oxygen supply. The oxygen concentrator module can purify, separate, and concentrate oxygen in air and can simultaneously produce the oxygen at pressure. The oxygen concentrator module can minimize hardware mass, volume, and power while performing at high reliability over a range of inlet pressure and oxygen concentration and delivering a range of outlet flow and concentration to the patient. The solid state technology of the oxygen concentrator module can comprise a portable oxygen concentration stack and supporting electrical, control, and fluids systems. The core technology is inherently capable of flow turndown and can be responsive to changing demands, making it easily integrated with application feedback systems. The power consumption can be directly proportional to the flow of concentrated oxygen and the volume footprint is low compared to other commercial technologies.

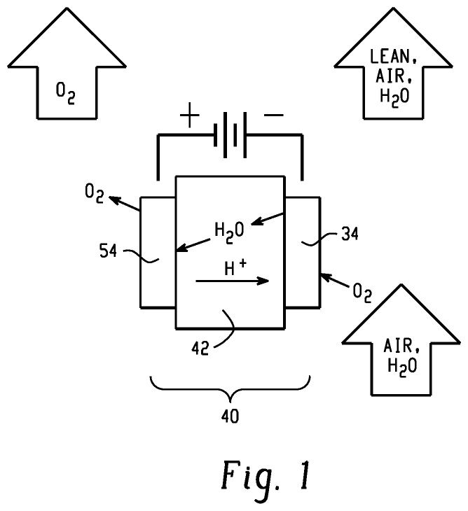

The oxygen concentrator module includes an electrochemical cell that can be a combination of a water electrolyzer and a fuel cell. As is illustrated in FIG. 1, a humid air stream (indicated by the Air, H₂O arrow) can be directed to a cathode chamber of the electrochemical cell 40. The water vapor can permeate through the proton exchange membrane 42 to the dry anode side of the electrochemical cell. A direct current electrical potential can be applied to the electrochemical cell and the water can be electrolyzed on the anode 54 creating an oxygen product stream (indicated by the O₂ arrow). The positively charged protons that are generated from electrolysis can permeate through the membrane 42 to the negatively charged cathode 34, where they can recombine with oxygen from the air to recreate water. The oxygen concentrator module can include more than one electrochemical cell based on the output of oxygen desired. Since oxygen can be consumed and generated at equivalent rates, the oxygen can be concentrated and delivered to a patient or to storage. Water vapor from the ambient air can be consumed and regenerated with no net change in the environmental humidity. A key aspect of this design can be considered to be that management of water can be achieved within the cell, such that performance can be maximized and power can be minimized

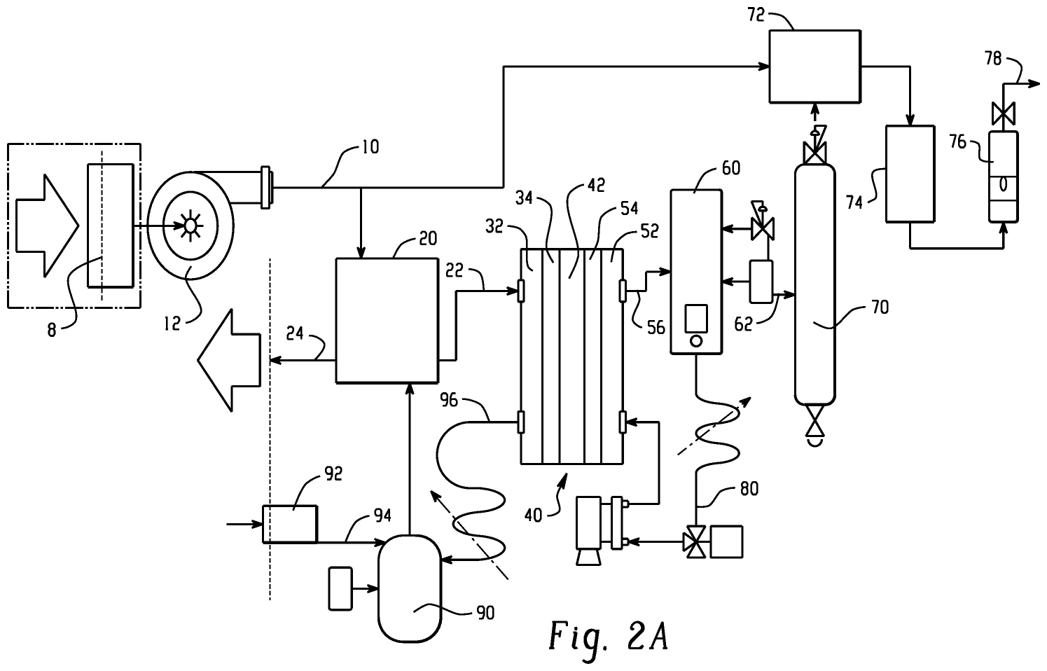

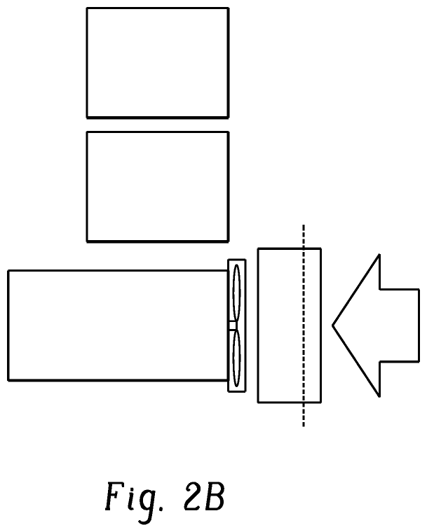

FIG. 2A is an illustration of an aspect of the oxygen concentrator module. FIG. 2A illustrates that a gas feed stream 10 can be in fluid communication with an electrochemical cell 40. The gas feed stream 10 can be introduced to the system, for example, via at least one of blower 12, an air compressor, a compressed gas tank, a pump, or the like. One or more filters 8 can be located upstream of the electrochemical cell 40 and can filter the gas feed stream 10 prior to introduction to the electrochemical cell 40. The gas feed stream 10 can comprise humid air. The gas feed stream 10 can comprise nitrogen, oxygen, carbon dioxide, and water. The gas feed stream 10 can comprise less than or equal to 22 volume percent, or 5 to 21 volume percent, or 5 to 15 volume percent of oxygen based on the total volume of the gas feed stream.

All or a portion of the gas feed stream 10 can be directed to the electrochemical cell 40. Prior to introduction to the electrochemical cell 40, all or a portion of the gas feed stream 10 can be directed to an enthalpy exchanger 20. An exchanged stream 22 comprising all or a portion of the gas feed stream 10 can be directed to a cathode chamber 32 of the electrochemical cell 40. An air outlet stream 24 can be removed from the enthalpy exchanger 20.

The water vapor from the gas feed stream 10 can permeate through the proton exchange membrane 42 to the anode 54. The water can be electrolyzed at the anode 54 to create oxygen according to reaction (1).

$${H}_{2} O \to \frac{1}{2} {O}_{2} + 2 {e}^{-} + 2 {H}^{+} \qquad ( 1 )$$

The protons formed at the anode 54 can permeate through the proton exchange membrane 42 back to the cathode 34 where they can combine with oxygen to recreate water via the reverse reaction (2).

$$\frac{1}{2} {O}_{2} + 2 {e}^{-} + 2 {H}^{+} \to {H}_{2} O \qquad ( 2 )$$

The product oxygen can be collected in the anode side chamber 52 and a concentrated oxygen stream 56 can be produced. The concentrated oxygen stream 56 can comprise greater than or equal to 25 volume percent, or 25 to 100 volume percent, 50 to 99 volume percent, 60 to 85 volume percent of oxygen based on the total volume of the concentrated oxygen stream 56. The concentrated oxygen stream 56 can be directed to a patient. The concentrated oxygen stream 56 can be in fluid communication with an oxygen storage tank 70. The concentrated oxygen stream 56 can be in fluid communication with a drier 60. The drier 60 can be located upstream of, for example, the oxygen storage tank 70 or a patient. The drier 60 can remove water from the concentrated oxygen stream to form a dry oxygen stream 62. The drier 60 can include at least one of a condenser, a membrane separator, a heat exchanger, or the like. A portion of the dry oxygen stream 62 can be directed back to the drier 60 for further drying. All or a portion of the dry oxygen stream can be removed from the drier 60. A back pressure regulator can regulate the flow out of the drier 60 and a valve can direct the flow back to drier 60 and/or away from the drier 60.

At least a portion of the concentrated oxygen stream 56 that is optionally dried can be mixed with a portion of the gas feed stream 10 in mixer 72. This mixing can be used to adjust the oxygen concentration of the resultant stream to a predetermined oxygen content.

At least a portion of the concentrated oxygen stream 56 that is optionally dried or mixed with the gas feed stream 10 can be in fluid communication with a filter 74, for example, with a filter 74, such as a high-efficiency particulate air (HEPA) filter. A flow rate of the output stream 78 can be adjusted via flow adjuster 76. The concentrated oxygen stream 56, for example, as output stream 78 can comprise 20 to 90 volume percent, or 25 to 90 volume percent, or 35 to 90 volume percent of oxygen based on the total volume of the output stream 78.

A water recycle stream 80 can be produced by the drier 60. All or a portion of the water recycle stream 80 can be in fluid communication with the electrochemical cell 40 via introduction on either or both of the cathode side chamber 32 or the anode side chamber 52. All or a portion of the water recycle stream 80 can be in fluid communication with the cathode side chamber 32 optionally via at least one of a water storage tank 90 or the enthalpy exchanger 20. All or a portion of the water recycle stream 80 can be in fluid communication with the anode side chamber 52 optionally via the water storage tank 90.

The water storage tank 90 can store one or both of the water from the water recycle stream 80 or from a fresh water stream 94. The water storage tank 90 can store the water and introduce the water to the electrochemical cell 40 in a controlled manner The fresh water stream 94 from a fresh water source can be in fluid communication with a water filter 92 prior to entering the oxygen concentrator module. A phase separator capable of converting the liquid water to a gas can be located downstream of the water storage tank 90 and upstream of the enthalpy exchanger 20.

A separated water stream 96 can be formed in the cathode side chamber 32 and can be withdrawn. The separated water stream 96 can be in fluid communication with the water tank 90.

FIG. 2B is an illustration of an illustrative unit of FIG. 2A in the dashed box for introducing the gas feed stream 10. The unit can include a filter 8 powered by a stack having, for example, a stack power supply (illustrated by the bottom-most box), a 24-volt direct current (illustrated by the middle box), and that is operated by a Graphical User Interface (GUI) controller (illustrated by the top-most box).

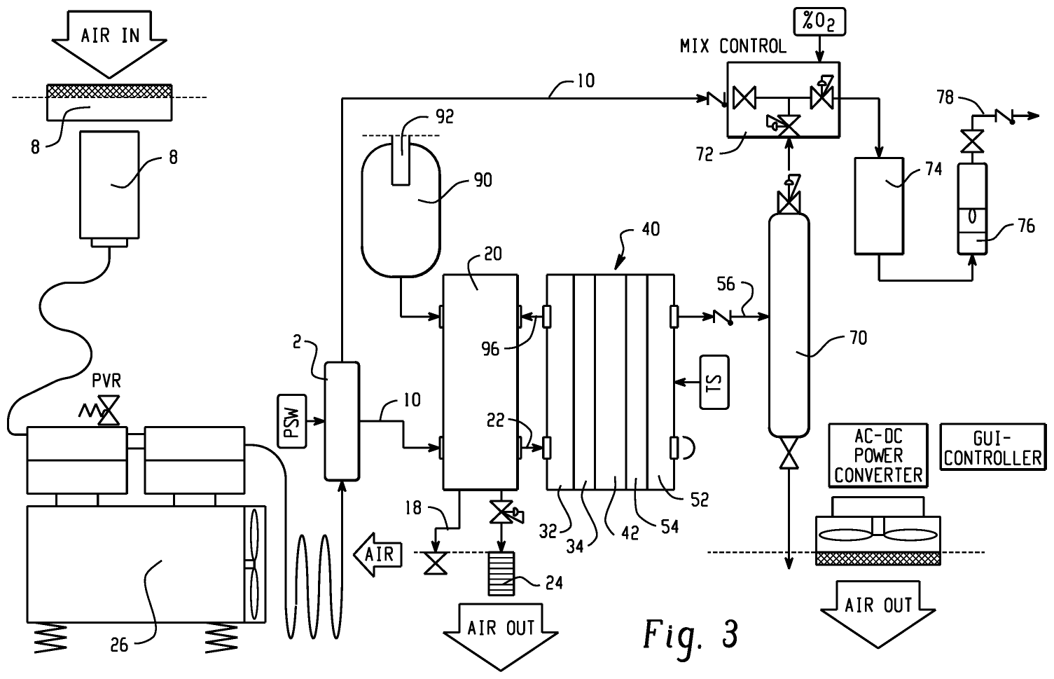

FIG. 3 is an illustration of an aspect of the oxygen concentrator module. FIG. 3 illustrates that a gas feed stream 10 can be in fluid communication with an electrochemical cell 40. The gas feed stream 10 can be introduced to the system, for example, via at least one of an air compressor 26, a blower, a compressed gas tank 2, a pump, or the like. One or more filters 8 can be located upstream of the electrochemical cell 40 and can filter the gas feed stream 10 prior to introduction to the electrochemical cell 40. The gas feed stream 10 can comprise humid air. The gas feed stream 10 can comprise nitrogen, oxygen, carbon dioxide, and water. The gas feed stream 10 can comprise less than or equal to 22 volume percent, or 5 to 21 volume percent, or 5 to 15 volume percent of oxygen based on the total volume of the gas feed stream.

All or a portion of the gas feed stream 10 can be directed to the electrochemical cell 40. Prior to introduction to the electrochemical cell 40, all or a portion of the gas feed stream 10 can be directed to an enthalpy exchanger 20. The enthalpy exchanger 20 can be in a stack exchanger. An exchanged stream 22 comprising all or a portion of the gas feed stream 10 can be directed to a cathode chamber 32 of the electrochemical cell 40. An air outlet stream 24 can be removed from the enthalpy exchanger 20. A water bleed stream 18 can be removed from the enthalpy exchanger 20.

The water vapor from the gas feed stream 10 can permeate through the proton exchange membrane 42 to the anode 54. The water can be electrolyzed at the anode 54 to create oxygen according to reaction (1).

$${H}_{2} O \to \frac{1}{2} {O}_{2} + 2 {e}^{-} + 2 {H}^{+} \qquad ( 1 )$$

The protons formed at the anode 54 can permeate through the proton exchange membrane 42 back to the cathode 34 where they can combine with oxygen to recreate water via the reverse reaction (2).

$$\frac{1}{2} {O}_{2} + 2 {e}^{-} + 2 {H}^{+} \to {H}_{2} O \qquad ( 2 )$$

The product oxygen can be collected in the anode side chamber 52 and a concentrated oxygen stream 56 can be produced. The concentrated oxygen stream 56 can be directed to a patient. The concentrated oxygen stream 56 can be in fluid communication with an oxygen storage tank 70. The concentrated oxygen stream 56 can optionally be in fluid communication with a drier. The drier can be located upstream of, for example, the oxygen storage tank 70 or a patient. The drier can remove water from the concentrated oxygen stream to form a dry oxygen stream. The drier can include at least one of a condenser, a membrane separator, a heat exchanger, or the like.

At least a portion of the concentrated oxygen stream 56 can be mixed with a portion of the gas feed stream 10 in mixer 72. This mixing can be used to adjust the oxygen concentration of the resultant stream to a predetermined oxygen content.

At least a portion of the concentrated oxygen stream 56 that is optionally mixed with the gas feed stream 10 can be in fluid communication with a filter 74, for example, with a filter 74, such as a HEPA filter. A flow rate of the output stream 78 can be adjusted via flow adjuster 76. The concentrated oxygen stream 56, for example, as output stream 78 can comprise 20 to 90 volume percent, or 25 to 90 volume percent, or 35 to 90 volume percent of oxygen based on the total volume of the output stream 78.

A water storage tank 90 can supply water to the oxygen concentrator module from a fresh water stream. The water storage tank 90 can store the water and introduce the water to the electrochemical cell 40 in a controlled manner The fresh water stream from a fresh water source can be in fluid communication with a water filter prior to entering the oxygen concentrator module.

A separated water stream 96 can be formed in the cathode side chamber 32 and can be withdrawn. The separated water stream 96 can be in fluid communication with the enthalpy exchanger 20.

The oxygen concentrator module can be portable, weighing less than 23 kilograms (kg), or 5 to 15 kg, or 5 to 10 kg. The oxygen concentrator module can produce as much as 5.5 liters per minute of pure oxygen, for example, based on 52 liters per minute of air input. The oxygen concentrator module can produce an oxygen output pressure of 5 bara. Product oxygen can be produced dry or saturated with water. The system can have a power requirement of less than or equal to 1.3 kilowatts (kW), or 0.5 to 1.3 kW. It is noted that these values can depend on the module design and that the oxygen concentrator module can likewise be configured to provide higher or lower amounts of the final oxygen and at different pressures as desired.

The oxygen concentrator module can comprise an electrochemical cell 40 comprising a cathode 34, an anode 54, a proton exchange membrane 42 located in between the cathode 34 and the anode 54, a cathode side chamber 32 located on a side of the cathode 34 opposite the proton exchange membrane 42, and an anode side chamber 52 located on a side of the anode 54 opposite the proton exchange membrane 42; a gas feed stream 10 in fluid communication with the cathode side chamber 32; a concentrated oxygen stream 56 in fluid communication with the anode side chamber 52 to remove the concentrated oxygen stream 56 from the anode side chamber 52; a separated water stream 96 in fluid communication with the cathode side chamber 32 to remove the separated water stream 96 from the cathode side chamber 32; and an enthalpy exchanger 20 in fluid communication with the cathode side chamber 32 via an exchanged stream 22, wherein the gas feed stream 10 is in fluid communication with the enthalpy exchanger 20 upstream of the electrochemical cell 40 to form the exchanged stream.

The enthalpy exchanger 20 can be a stack exchanger and the oxygen concentrator module can comprise a stack of the electrochemical cell 40 and the stack exchanger. The stack can include one or more electrochemical cells 40. The oxygen concentrator module can comprise a drier 60 that is in fluid communication with the anode side chamber 52 via the concentrated oxygen stream 56 and a water recycle stream 80 can be in fluid communication with the drier 60 and one or both of the enthalpy exchanger 20 or the anode side chamber 52. The oxygen concentrator module can comprise a water tank 90 that is in fluid communication with the enthalpy exchanger 20. The oxygen concentrator module can comprise a mixer 72 that can be in fluid communication with the gas feed stream 10 and the concentrated oxygen stream and a flow rate of the respective streams can be adjustable such that an oxygen concentration of an output stream 78 can be adjusted. The oxygen concentrator module can comprise an oxygen tank 70 in fluid communication with the anode side chamber 52. The oxygen concentrator module can comprise at least one of a blower 12, an air compressor 26, a compressed gas tank 2, or a pump configured to supply the cathode side chamber with the gas feed stream 10. The oxygen concentrator module can comprise one or more filters to purify at least one of the gas feed stream 10 or a water supply. The oxygen concentrator module can have a power requirement of less than or equal to 1.3 kW, or 0.5 to 1.3 kW.

The method of concentrating oxygen comprises using the oxygen concentrator module disclosed herein to concentrate oxygen from air. The method can comprise introducing a gas feed stream 10 to a cathode side chamber 32 of an electrochemical cell 40 comprising a cathode 34, an anode 54, a proton exchange membrane 42 located in between the cathode 34 and the anode 54, the cathode side chamber 32 located on a side of the cathode 34 opposite the proton exchange membrane 42, and an anode side chamber 52 located on a side of the anode 54 opposite the proton exchange membrane 42; removing a concentrated oxygen stream 56 from the anode side chamber 52; removing a separated water stream 96 from the cathode side chamber 32; and directing the gas feed stream 10 to an enthalpy exchanger 20 upstream of the electrochemical cell 40 and hydrating the gas feed stream 10 in the enthalpy exchanger.

The method can comprise drying the concentrated oxygen stream 56 in a drier 60 and the hydrating the gas feed stream 10 can comprise hydrating the gas feed stream 10 with a water recycle stream 80 from the drier 60. The hydrating the gas feed stream 10 can comprise hydrating the gas feed stream 10 with a water stream from a water tank 90. The method can comprise reducing an oxygen concentration of the concentrated oxygen stream (56) by mixing the concentrated oxygen stream (56) with a portion of the gas feed stream (10). The method can comprise directing the concentrated oxygen stream to an oxygen storage tank (70). The method can comprise filtering one or more of the gas feed stream 10 or the concentrated oxygen stream 56. The method can produce as much as 5.5 liters per minute of pure oxygen, or, in other words, the removing the concentrated oxygen stream 56 can remove as much as 5.5 liters per minute of pure oxygen from the oxygen concentrator module.

The compositions, methods, and articles can alternatively comprise, consist of, or consist essentially of, any appropriate materials, steps, or components herein disclosed. The compositions, methods, and articles can additionally, or alternatively, be formulated so as to be devoid, or substantially free, of any materials (or species), steps, or components, that are otherwise not necessary to the achievement of the function or objectives of the compositions, methods, and articles.

As used herein, “a,” “an,” “the,” and “at least one” do not denote a limitation of quantity, and are intended to cover both the singular and plural, unless the context clearly indicates otherwise. For example, “an element” has the same meaning as “at least one element,” unless the context clearly indicates otherwise. The term “combination” is inclusive of blends, mixtures, alloys, reaction products, and the like. Also, “at least one of” means that the list is inclusive of each element individually, as well as combinations of two or more elements of the list, and combinations of at least one element of the list with like elements not named.

The term “or” means “and/or” unless clearly indicated otherwise by context. Reference throughout the specification to “an aspect”, “another aspect”, “some aspects”, and so forth, means that a particular element (e.g., feature, structure, step, or characteristic) described in connection with the aspect is included in at least one aspect described herein, and may or may not be present in other aspects. In addition, it is to be understood that the described elements may be combined in any suitable manner in the various aspects.

The endpoints of all ranges directed to the same component or property are inclusive of the endpoints, are independently combinable, and include all intermediate points and ranges. For example, ranges of “up to 25 vol %, or 5 to 20 vol %” is inclusive of the endpoints and all intermediate values of the ranges of “5 to 25 vol %,” such as 10 to 23 vol %, etc.

Unless defined otherwise, technical and scientific terms used herein have the same meaning as is commonly understood by one of skill in the art to which this disclosure belongs.

In the figures, various other equipment such as valves (such as pressure reducing valves), pumps, thermocouples, and pressure regulators, etc. can be present. TS stands for stack temperature.

All cited patents, patent applications, and other references are incorporated herein by reference in their entirety. However, if a term in the present application contradicts or conflicts with a term in the incorporated reference, the term from the present application takes precedence over the conflicting term from the incorporated reference.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

What is claimed is:

1. An oxygen concentrator module comprising:
an electrochemical cell (40) comprising a cathode (34), an anode (54), a proton exchange membrane (42) located in between the cathode (34) and the anode (54), a cathode side chamber (32) located on a side of the cathode (34) opposite the proton exchange membrane (42), and an anode side chamber (52) located on a side of the anode (54) opposite the proton exchange membrane (42);
a gas feed stream (10) in fluid communication with the cathode side chamber (32);
a concentrated oxygen stream (56) in fluid communication with the anode side chamber (52) to remove the concentrated oxygen stream (56) from the anode side chamber (52);
a separated water stream (96) in fluid communication with the cathode side chamber (32) to remove the separated water stream (96) from the cathode side chamber (32); and
an enthalpy exchanger (20) in fluid communication with the cathode side chamber (32) via an exchanged stream (22), wherein the gas feed stream (10) is in fluid communication with the enthalpy exchanger (20) upstream of the electrochemical cell (40) to form the exchanged stream.

2. The oxygen concentrator module of claim 1, wherein the enthalpy exchanger (20) is a stack exchanger and the oxygen concentrator module comprises a stack of the electrochemical cell (40) and the stack exchanger.

3. The oxygen concentrator module of any of the preceding claims, further comprising a drier (60) that is in fluid communication with the anode side chamber (52) via the concentrated oxygen stream (56); wherein a water recycle stream 80 is in fluid communication with the drier (60) and one or both of the enthalpy exchanger (20) or the anode side chamber (52).

4. The oxygen concentrator module of any of the preceding claims, further comprising a water tank (90) that is in fluid communication with the enthalpy exchanger (20).

5. The oxygen concentrator module of any of the preceding claims, further comprising a mixer (72); wherein the mixer is in fluid communication with the gas feed stream (10) and the concentrated oxygen stream; and wherein a flow rate of the respective streams is adjustable such that an oxygen concentration of an output stream (78) can be adjusted.

6. The oxygen concentrator module of any of the preceding claims, further comprising an oxygen take (70) in fluid communication with the anode side chamber (52).

7. The oxygen concentrator module of any of the preceding claims, further comprising at least one of a blower (12), an air compressor (26), a compressed gas tank (2), or a pump configured to supply the cathode side chamber with the gas feed stream (10).

8. The oxygen concentrator module of any of the preceding claims, further comprising one or more filters to purify at least one of the gas feed stream (10) or a water supply.

9. The oxygen concentrator module of any of the preceding claims, wherein the oxygen concentrator module has a power requirement of less than or equal to 1.3 kW, or 0.5 to 1.3 kW.

10. An method of concentrating oxygen comprising:
introducing a gas feed stream (10) to an cathode side chamber (32) of an electrochemical cell (40) comprising a cathode (34), an anode (54), a proton exchange membrane (42) located in between the cathode (34) and the anode (54), the cathode side chamber (32) located on a side of the cathode (34) opposite the proton exchange membrane (42), and an anode side chamber (52) located on a side of the anode (54) opposite the proton exchange membrane (42);
removing a concentrated oxygen stream (56) from the anode side chamber (52);
removing a separated water stream (96) from the cathode side chamber (32); and
directing the gas feed stream (10) to an enthalpy exchanger (20) upstream of the electrochemical cell (40) and hydrating the gas feed stream (10) in the enthalpy exchanger;
the method can use the oxygen concentrator module of any one of the preceding claims.

11. The method of claim 10, further comprising drying the concentrated oxygen stream (56) in a drier (60) and wherein the hydrating the gas feed stream (10) comprising hydrating the gas feed stream (10) with a water recycle stream (80) from the dried (60).

12. The method of any of claims 10 to 11, wherein the hydrating the gas feed stream (10) comprising hydrating the gas feed stream (10) with a water stream from a water tank (90).

13. The method of any of claims 10 to 12, further comprising reducing an oxygen concentration of the concentrated oxygen stream (56) by mixing the concentrated oxygen stream (56) with a portion of the gas feed stream (10).

14. The method of any of claims 10 to 13, further comprising directing the concentrated oxygen stream to an oxygen storage tank (70).

15. The method of any of claims 10 to 14, further comprising filtering one or more of the gas feed stream (10) or the concentrated oxygen stream (56).

16. The method of any of claims 10 to 15, wherein the removing the concentrated oxygen stream (56) removes as much as 5.5 liters per minute of pure oxygen.
